(19) United States
(12) Patent Application Publication
(10) Pub. No.: US 2026/0114910 A1
(43) Pub. Date: Apr. 30, 2026

(54) INTRAMEDULLARY NAIL WITH CONTINUOUS COMPRESSION

(71) Applicant: DePuy Synthes Products, Inc., Raynham, MA (US)

(72) Inventors: Ross Hamel, West Chester, PA (US); Glen J. Pierson, Glenmoore, PA (US); Rene Haag, Berwyn, PA (US)

(21) Appl. No.: 19/375,725

(22) Filed: Oct. 31, 2025

Related U.S. Application Data
(60) Provisional application No. 63/714,158, filed on Oct. 31, 2024.

(51) Int. Cl. A61B 17/72; A61B 17/00; A61B 17/68

(52) U.S. Cl. CPC A61B 17/7225; A61B 2017/00367; A61B 2017/681

(57) ABSTRACT

An intramedullary nail can extend along a central axis and include a first member, a second member, and a biasing member. The first member can be positioned in a medullary canal of a bone. The first member can define a first bone fixation hole configured to receive a first bone fixation element so as to couple the first member to a first bone portion. The second member can be inserted into the first member and can define a second bone fixation hole configured to receive a respective second bone fixation element so as to couple the second member to a second bone portion spaced from the first bone portion. The biasing member can apply a biasing force to one of the first and second members so as to decrease a distance from the first bone fixation hole to the second bone fixation hole, thereby creating a continuous compression force between the first and second bone members at their interface.

### CROSS-REFERENCE TO RELATED APPLICATIONS

This claims the benefit of U.S. Patent Application Ser. No. 63/714,158 filed Oct. 31, 2024, the disclosure of which is hereby incorporated by reference as if set forth in its entirety herein.

### TECHNICAL FIELD

The present disclosure relates generally to bone fixation devices, and more specifically to intramedullary nails to couple bone segments to each other.

### BACKGROUND

Conventional intramedullary nails are configured to be inserted into the medullary canal of a bone that has been fractured so as to define a proximal bone segment and a distal bone segment that is separated from the proximal bone segment by a bone gap. Conventional intramedullary nails are elongate along a substantially central longitudinal axis, and include bone anchor holes that extend through the nail along respective axes that are angularly offset, for instance perpendicular, with respect to the longitudinal axis of the intramedullary nail, and configured to receive bone anchors. The bone anchor holes can be divided into a proximal bone anchor holes that extend through the proximal end of the intramedullary nail and distal bone anchor holes that extend through the distal end of the intramedullary nail. Thus, the intramedullary nail can be inserted into the medullary canal of the fractured long bone such that the proximal bone anchor holes are aligned with the proximal bone segment and the distal bone anchor holes are aligned with the distal bone segment on opposite sides of the bone gap. The bone screws can be driven into the bone segments and the corresponding bone anchor holes so as to fasten the intramedullary nail to the fractured long bone and stabilize the proximal and distal bone segments relative to each other, thereby promoting healing.

Certain intramedullary nails include features that allow the proximal and distal bone segments to compress toward each other, thereby approximating the bone gap. For instance, screws can be inserted into the distal bone segment and the distal bone anchor holes of the intramedullary nail so as to fix the distal bone segment to the distal intramedullary nail, and a standing load can be applied to cause the proximal bone segment to translate relative to the intramedullary nail toward the distal bone segment. However, certain compression features, while facilitating the approximation of the bone gap, are not self-retaining. Accordingly, compression is maintained manually while fixing the distal bone segment to the distal end of the intramedullary nail. Other compression features are self-retaining so as to maintain approximation of the bone gap while the distal bone segment is fixed to the distal end of the intramedullary nail. However, self-retaining compression features typically add movable components in the intramedullary nail and are time consuming and complex to use.

### SUMMARY

An intramedullary nail that extends along a central axis can include a first member, a second member, and a biasing member. The first member can be configured to be positioned in a medullary canal of a bone, wherein the first member defines at least one first bone fixation hole configured to receive a respective at least one first bone fixation element so as to couple the first member to a first bone portion of the bone. The second member can be configured to be at least partially inserted into the first member, wherein the second member defines at least one second bone fixation hole configured to receive a respective at least one second bone fixation element so as to couple the second member to a second bone portion of the bone that can be spaced from the first bone portion. The biasing member can be adapted to apply a biasing force to at least one of the first and second members so as to decrease a distance from the at least one first bone fixation hole to the at least one second bone fixation hole along the central axis.

The first member can be fixed to the first bone portion. The first member can be fixed to the first bone portion by a first bone fixation element. The second member can be fixed to the second bone portion. The second member can be fixed to the second bone portion by a second bone fixation element. The first member includes a first slot that can be elongate along a longitudinal axis of the first member and the second bone fixation element can be positioned within the first slot such that the first member can be movable relative to the second bone fixation element. The second member includes a second slot that can be elongate along a longitudinal axis of the second member and the first bone fixation element can be positioned within the second slot such that the second member can be movable relative to the first bone fixation element. The first member includes a channel adapted to receive the biasing member.

A first end of the biasing member can be supported by the first member in the channel, and a second end of the biasing member bears against the second member in a select direction, and the biasing force can be applied to the second member in the select direction. The biasing member extends from a shoulder of the first member inside the channel to the second member in the select direction. The first member can include a channel adapted to receive the second member. The first member can include a channel adapted to receive the second member and the biasing member. The intramedullary nail can be configured to transition from an initial configuration to a loaded configuration, and from the loaded configuration to a compressed configuration. In the initial configuration a distal end of the first member can be spaced from a distal end of the second member by an initial distance. In the compressed configuration the distal end of the first member can be spaced from the distal end of the second member by a compressed distance. The initial distance can be greater than the compressed distance. A length of the intramedullary nail can be the same in the initial configuration and the compressed configuration, wherein the length can be measured along the central axis from a first terminal end of the first member to a second terminal end of the second member opposite the first terminal end. The biasing member can be compressed between the first member and the second member.

In a further embodiment, an actuator is adapted to move the second member relative to the first member against the biasing force of the biasing member. The actuator can be adapted to move the second member relative to the first member along the longitudinal axis. Rotational motion of the actuator can move the second member linearly relative to the first member. The actuator can be threadedly engaged with the first member. The actuator can be within the channel of the first member. The channel can be elongate along the longitudinal axis. Movement of the actuator in a first direction can transition the biasing member from an initial configuration to an energized configuration. Movement of the actuator in a second direction can allow the second member to move relative to the first member. The biasing force can be applied to the second member, and the actuator can be movable away from the second member so as to allow the biasing force to move the second member so as to decrease the distance between the at least one first bone fixation hole to the at least one second bone fixation hole. The second direction can be opposite the first direction. The first bone portion and the second bone portion are two pieces of a same bone. The first bone portion and the second bone portion can be different bones.

A method of assembling an intramedullary nail can include positioning a second member within a channel of a first member, engaging the first member with an actuator, and moving the first and second members relative to each other. The coupling step can include positioning the second member within a channel of the first member. The method can include positioning a first fixation element in a first slot of the first member and a second bone fixation hole of the second member. The method can include positioning a second fixation element in a first bone fixation hole of the first member and a second slot of the second member. The method can include actuating the actuator thereby transitioning a biasing member from a first state to a second state. The intramedullary nail can be elongate along a longitudinal axis and transitioning the biasing member can include threadedly advancing the actuator along the longitudinal axis.

### BRIEF DESCRIPTION OF THE DRAWINGS

The present application is further understood when read in conjunction with the appended drawings. For the purpose of illustrating the subject matter, there are shown in the drawings exemplary aspects of the subject matter; however, the presently disclosed subject matter is not limited to the specific methods, devices, and systems disclosed. In the drawings:

FIG. 1 is a perspective view of an intramedullary nail in one example shown positioned within a bone;

FIG. 2 is a sectional view of the intramedullary nail of FIG. 1, but shown in a final compressed configuration;

FIG. 3 is a sectional view of the intramedullary nail of FIG. 1 shown in an initial configuration with a distal bone anchor implanted;

FIG. 4 is a sectional view of the intramedullary nail of FIG. 3 shown in a loaded configuration;

FIG. 5 is a sectional view of the intramedullary nail of FIG. 4, but showing the bone portions reduced;

FIG. 6 is a sectional view of the intramedullary nail of FIG. 5, but showing proximal bone anchors implanted prior to iterating the intramedullary nail to the final compressed configuration of FIG. 1; and

FIG. 7 is a flow chart of a method of assembling the fixation device of FIG. 1 in one example.

Aspects of the disclosure will now be described in detail with reference to the drawings, wherein like reference numbers refer to like elements throughout, unless specified otherwise.

### DETAILED DESCRIPTION

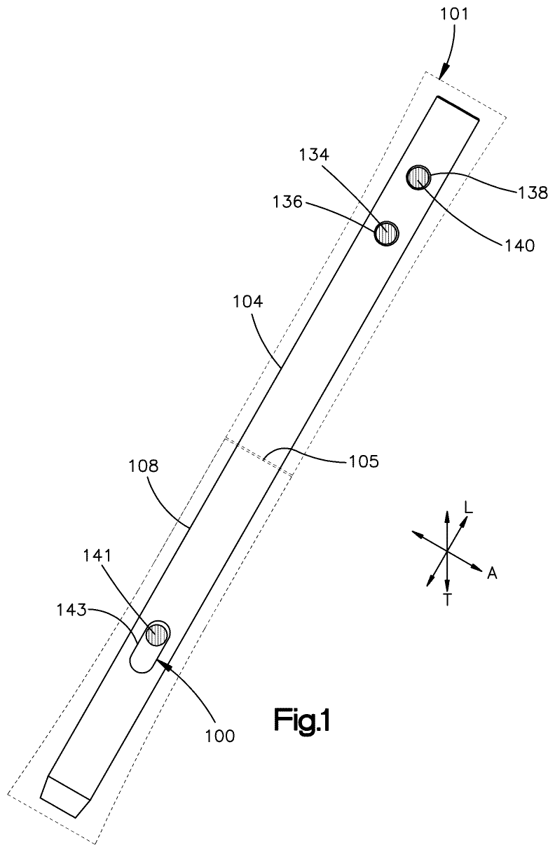

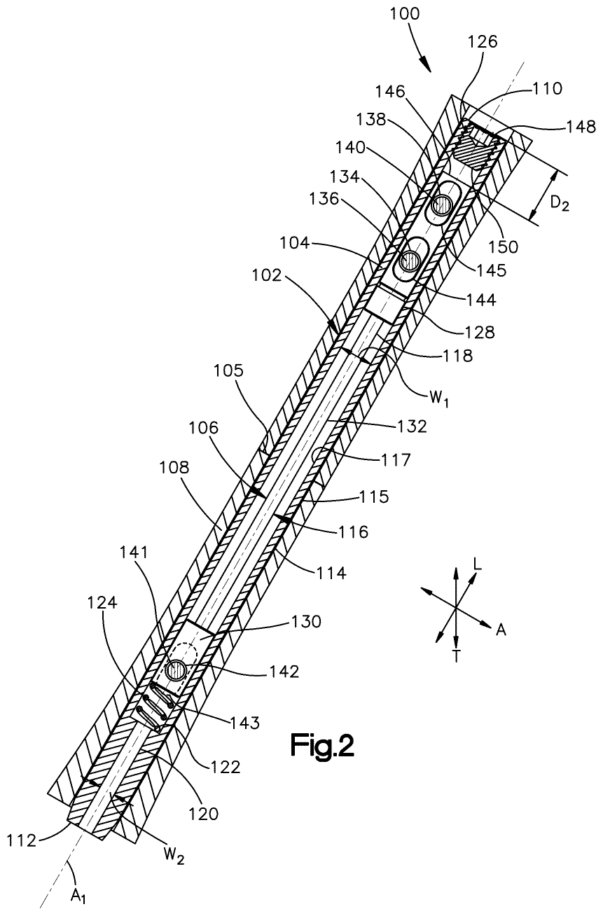

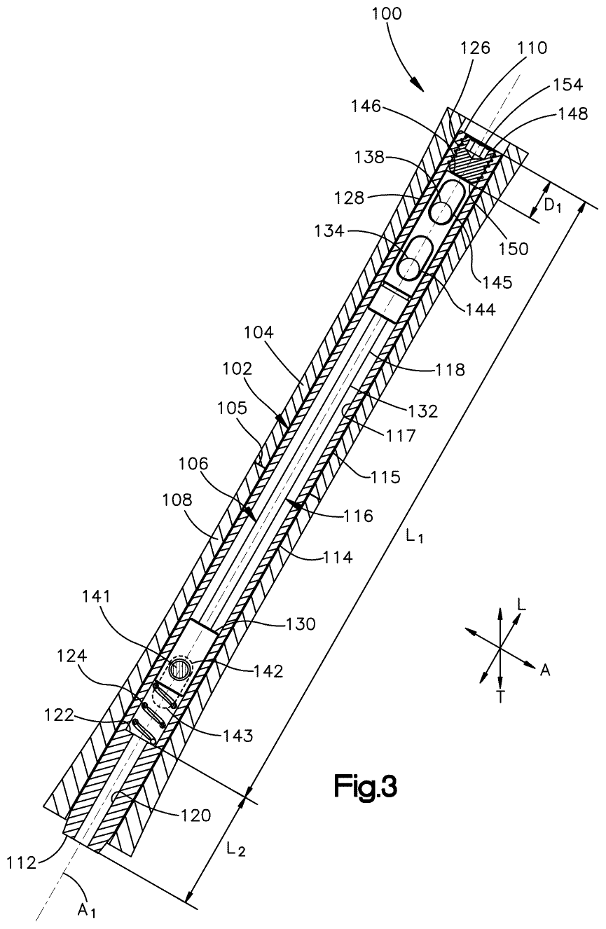

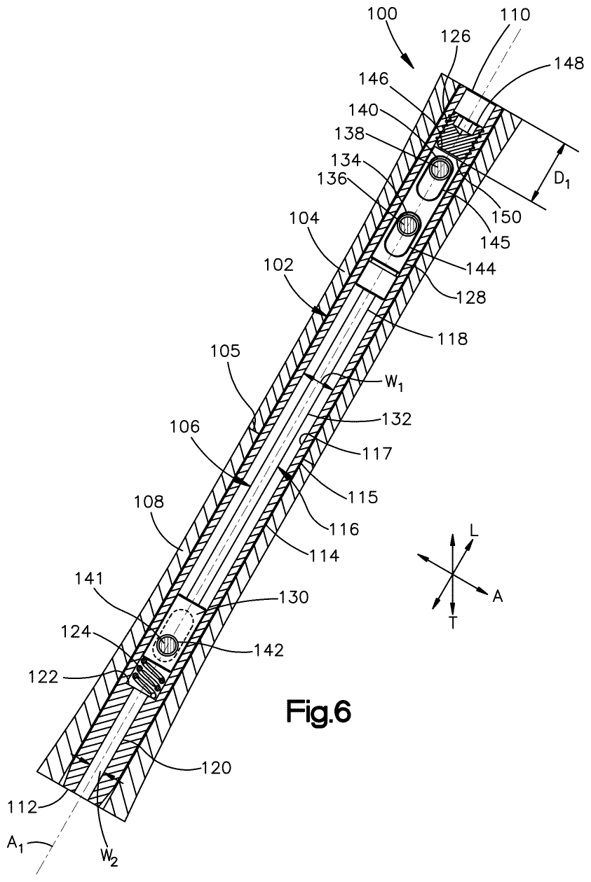

Referring to FIG. 1, a bone fixation device 100 is configured to be positioned within a bone 101. The bone fixation device 100 can be adapted to couple first and second portions 104 and 108 of bone to each other. The bone fixation device 100 can be adapted to urge the bone portions 104 and 108 toward each other. The bone fixation device 100 can provide a force that compresses the bone portions 104 and 108 against each other. The bone fixation device 100 can define a compressed configuration that causes the bone portions 104 and 108 to exert a compressive force against each other. The bone fixation device 100 can provide a constant compressive force after it is implanted and iterated from an initial configuration (FIG. 3) to a loaded configuration (FIG. 6) whereby a biasing member 124 is loaded to apply a compression force, to a compressed configuration (FIG. 2) whereby the biasing member applies the force that causes fixation device 100 to maintain a compressive force across the first and second bone portions 104 and 108.

The bone fixation device 100 can be adapted to be positioned in an medullary canal in the initial configuration. Thus, the bone fixation device 100 can be referred to as an intramedullary nail. The bone portions can be coupled to the bone fixation device 100 to maintain the position of the bone portions relative to each other in the compressed configuration, whereby the bone portions are compressed against each other, so as to promote bone healing.

In some examples, the bone portions 104 and 108 can be defined by a single fractured bone. The bone can be a long bone. For instance, the bone can be a tibia, fibula, femur, metatarsal, phalange, humerus, radius, ulna, metacarpal, or any suitable alternative bone as desired. In other examples, the bone portions 104 and 108 can defined by be two different bones that are coupled together, for instance during a fusion procedure.

Referring to now to FIG. 2, the bone fixation device 100 can be iterated to a compressed configuration whereby the bone fixation device provides a compression force against either or both of the first or second bone portions 104 and 108 that provides compression of the first and/or second bone portions 104 and 108 against each other. The fixation device 100 and operation of the fixation device 100 will now be described.

The fixation device 100 can define a first or proximal end 110 and a second or distal end 112 spaced from the proximal end 110 along a longitudinal direction L. For instance, the first end 110 can be opposite the second end 112 along the longitudinal direction L. The proximal end 110 can be spaced from the distal end 112 in a proximal direction. Thus, reference herein to a proximal direction or derivatives thereof can refer to a direction from the proximal end 110 toward the distal end 112. Conversely, the distal end 112 can be spaced from the proximal end 110 in a distal direction opposite the proximal direction. Thus, reference herein to a distal direction or derivatives thereof can refer to a direction from the distal end 112 toward the proximal end 110. The proximal direction and the distal direction can be opposite each other along the longitudinal direction L. In this regard, the distal direction can be referred to as a first direction. The proximal direction can be referred to as a second direction. In the illustrated example, the first bone portion 104 can be a proximal bone portion, and the second bone portion 108 can be a distal bone portion adjacent the proximal bone portion in the distal direction.

The bone fixation device 100 can include central axis A₁ that is oriented along the longitudinal direction L. Thus, the proximal end 110 and the distal end 112 of the bone fixation device 100 can be opposite each other along the central axis A₁. The fixation device 100 can be elongate along the central axis A₁. The bone fixation device 100 can include a first or outer member 102 that is configured to be coupled to a first bone portion 104. The bone fixation device 100 can further include a second or inner member 106 that is configured to be coupled to a second bone portion 108. The inner member 106 can be disposed in the outer member 102.

As will be described in more detail below, either or both of the outer member 102 and the inner member 106 can be movable relative to the other of the outer member 102 and the inner member 106 so as to cause the bone fixation device 100 to iterate from the initial configuration to the compressed configuration. For instance, either or both of the outer member 102 and inner member 106 can be movable relative to the other of the outer member 102 and inner member 106, respectively, in the longitudinal direction L from the initial configuration to the compressed configuration. In one example, the outer member 102 can be fixed to the first bone portion 104 and movable relative to the second bone portion 108. Similarly, the inner member 106 can be fixed to the second bone portion 108 and movable relative to the first bone portion 104 and fixed relative to the second bone portion 108. Either or both of the first bone portion 104 and the second bone portion 108 can thus move relative to the other of the first bone portion 104 and the second bone portion 108, respectively, as the respective either or both of the outer member 102 and inner member 106 moves relative to the other of the outer member 102 and the inner member 106, respectively.

The outer member 102 can be adapted to be positioned in an intramedullary canal. The outer member 102 can be positioned in an intramedullary canal of the first bone portion 104 and the second bone portion 108. Thus, the outer member 102 can extend from the first bone 104 to the second bone 108. The outer member 102 can include an outer wall 114. At least a portion of the outer wall 114 can be annular about the central axis A₁. The outer wall 114 can receive the inner member 106. The outer wall 114 can have a cylindrical cross-sectional shape taken along a plane oriented perpendicular to the longitudinal direction L. In other examples, the outer wall 114 can have an acylindrical cross-sectional shape. The cross-sectional plane can extend in a transverse direction T and a lateral direction A. The transverse direction T can be perpendicular to the longitudinal direction L, and the lateral direction A can be perpendicular to each of the longitudinal direction L and the transverse direction T. In some examples, the lateral direction A, the transverse direction T, and all other directions perpendicular to the longitudinal direction can define a radial direction that extends out from the central axis A₁.

The outer member 102 can be coupled to the first bone portion 104. For instance, the outer member 102 can be fixed to the first bone portion 104 such that relative motion between the outer member 102 and the first bone portion 104 is prevented. In one example, the outer member 102 can be fixed to the first bone portion 104 while allowing movement of the outer member 102 with respect to the second bone portion 108. Similarly, the inner member 106 can be fixed to the second bond portion 108 while allowing movement of the inner member 106 with respect to the first bone portion. In particular, as will now be described, the outer and inner members 102 and 106 can be fixed to the first and second bone portions 102 and 108, respectively, by respective bone anchors.

The outer member 102 can define at least one outer member aperture that is configured to receive a respective outer member bone anchor that fixes the outer member 102 to the first bone portion 104. In one example, the outer member can define first and second outer member apertures 134 and 138 configured to receive first and second outer member bone anchors 136 and 140, respectively, that are in turn fixed to the bone, and in particular to the first bone portion 104. Each of the first and second bone anchors 136 and 140 can be configured as a threaded bone fastener or bone screw, pin, rivet, nail, or adhesive. The first and second bone anchors 136 and 140 can be self-drilling in one example. The first and second bone anchors 136 and 140 can be locking screws. The first and second outer member bone anchors 136 and 140 can be referred to as first and second bone anchors 136 and 140 of the fixation device 100. Similarly, the first and second outer member apertures 134 and 138 can be referred to as first and second apertures of the fixation device 100.

As described above, the first and second outer member apertures 134 and 138 can be configured to receive the first and second bone anchors 136 and 140, and can thus be referred to as first and second bone fixation holes of the fixation device 100. The first and second outer member apertures 134 and 138 can extend through the outer wall 114 of the outer member 102 from an outer surface 115 of the outer wall 114 toward an inner surface 117 of the outer wall 114 that is opposite the outer surface 115. The inner surface 117 can face the central axis A₁, and the outer surface 115 can face away from the central axis A₁. The inner surface 117 can define an internal channel 116 of the outer member 102 that receives the inner member 106. Thus, the inner surface 117 can also face the inner member 106 of the fixation device 100. The apertures 134 and 138 can each define a first aperture end at the outer surface 115 and a second aperture end opposite the first aperture end along an aperture central axis. The second aperture end can be at the inner surface 117 of the outer wall 114. The first and second outer member apertures 134 and 138 can extend through the outer wall 114 at a respective first location of the outer wall 114, extend through the channel 116, and can extend through the outer wall 114 at a respective second location of the outer wall 114 that is opposite the respective first location, for instance with respect to the central axis A₁.

The apertures 134 and 138 can extend along respective first and second central axes in any suitable direction perpendicular to the longitudinal direction L. For instance, the apertures 134 and 138 can extend along the respective aperture central axes in the lateral direction A, the transverse direction T, or any other direction perpendicular to the longitudinal direction L. The apertures 134 and 138 can extend along the respective aperture central axes from the outer surface 115 of the outer wall 114 to the inner surface 117 of the outer wall 114. The aperture central axes of the apertures 134 and 138 can be parallel to each other or angularly offset with respect to each other. For instance, the apertures 134 and 138 can be aligned with each other along the central axis A₁ or the longitudinal direction L. Alternatively, the apertures 134 and 138 can be offset with respect to each other about the central axis A₁.

The first outer member aperture 134 can be configured to receive the first bone anchor 136. In particular, the first bone anchor 136 can be driven through the first bone portion 104 and into the first outer member aperture 134 so as to fix the outer member 102 to the first bone portion 104 with respect to relative movement along the longitudinal direction L. In particular, the first outer member bone anchor 136 can be driven at least into or through the first outer member aperture 134. The first outer member bone anchor 136 can extend from the first end of the first outer member aperture 134 toward the second end of the first outer member aperture 134. The first outer member bone anchor 136 can extend through the first aperture 134, and thus through the second end of the first aperture 134. The first outer member bone anchor 136 can extend from the outer surface 115 of the outer wall 114 toward the central axis A₁. For instance, the first outer member bone anchor 136 can extend to the central axis A₁. The first outer member bone anchor 136 can thus extend through the outer wall 114 of the outer member 102. The first outer member bone anchor 136 can be elongate along a respective central axis that is coincident with the respective central axis of the first outer member aperture 134 when the first outer member bone anchor 136 is driven into or through the first outer member aperture 134. The first bone anchor central axis can thus be perpendicular to the central axis A₁ or longitudinal direction L when the first outer member bone anchor 136 is disposed in the first outer member aperture 134.

The first outer member bone anchor 136 can threadedly engage the outer member 102 when the bone anchor 136 is driven into the aperture 134. In particular, the bone anchor 136 can threadedly engage an internal sidewall of the outer member 102 that defines the first aperture 134. The first aperture 134 can be sized and shaped to receive the first bone anchor 136 such that the outer member 102 is substantially positionally fixed to the first bone portion 104 with respect to relative movement along the longitudinal direction L, and in some examples with respect to any relative movement. The first aperture 134 can have a length in the longitudinal direction L that is about equal to an outer diameter of a threaded shaft of the first bone anchor 136 that threadedly engages the outer member.

As described above, the second outer member aperture 138 is configured to receive the second outer member bone anchor 140. The first and second apertures 134 and 138 can be identically sized and shaped. The second outer member bone anchor 140 can thus also be identical to the first outer member bone anchor 136 in size and shape. Thus, the first outer member bone anchor 136 and second outer member bone anchor 140 can be interchangeable. Alternatively, the apertures 134 and 138 and the respective bone anchors 136 and 140 can be differently sized and/or shaped as desired. The second outer member aperture 138 can be spaced from the first outer member aperture 134 in the longitudinal direction L. For instance, the second outer member aperture 138 can be spaced from the first outer member aperture 134 in the proximal direction. Thus, the first outer member aperture 134 can be referred to as a distal outer member aperture, and the second outer member aperture 138 can be referred to as a proximal outer member aperture.

The second outer member aperture 138 can be configured to receive the second outer member bone anchor 140. In particular, the second outer member bone anchor 140 can be driven through the first bone portion 104 and into the second outer member aperture 138 so as to fix the outer member 102 to the first bone portion 104 with respect to relative movement along the longitudinal direction L. In particular, the second outer member bone anchor 140 can be driven at least into or through the second outer member aperture 138. The second outer member bone anchor 140 can extend from the first end of the second outer member aperture 138 toward the second end of the second outer member aperture 138. The second outer member bone anchor 140 can extend through the second aperture 138, and thus through the second end of the second aperture 138. The second outer member bone anchor 140 can extend from the outer surface 115 of the outer wall 114 toward the central axis A₁. For instance, the second outer member bone anchor 140 can extend to the central axis A₁. The second outer member bone anchor 140 can thus extend through the outer wall 114 of the outer member 102. The second outer member bone anchor 140 can be elongate along a respective second central axis that is coincident with the respective second central axis of the second outer member aperture 134 when the second outer member bone anchor 140 is driven into or through the second outer member aperture 138. The second bone anchor central axis can thus be perpendicular to the central axis A₁ or longitudinal direction L when the second outer member bone anchor 140 is disposed in the second outer member aperture 138.

The second outer member bone anchor 140 can threadedly engage the outer member 102 when the bone anchor 140 is driven into the second outer member aperture 138. In particular, the bone anchor 140 can threadedly engage an internal sidewall of the outer member 102 that defines the second outer member aperture 138. The aperture 138 can be sized and shaped to receive the bone anchor 140 such that the outer member 102 is substantially positionally fixed to the first bone portion with respect to relative movement along the longitudinal direction L, and in some examples with respect to any relative movement. The second outer member aperture 138 can have a length in the longitudinal direction L that is substantially equal to an outer diameter of a threaded shaft of the second outer member bone anchor 140 that threadedly engages the outer member 102. While the outer member 102 is shown as defining first and second outer member apertures 134 and 138 that are configured to receive respective bone anchors that fix the outer member 102 to the first portion 104 of the bone, it should be appreciated that the outer member 102 can include any number of outer member apertures as desired.

It should be appreciated that the first and second outer member bone anchors 136 and 140 can be driven through respective first locations of the first bone portion 104, through the apertures 134 and 138 at the respective first locations of the outer wall 114, through the internal channel, through the apertures 134 and 138 at the respective second locations of the outer wall 114, and into respective second locations of the first bone portion 104. Thus, each of the first and second outer member bone anchors 136 and 140 can be secured to the first and second locations of the first bone portion 104.

The inner member 106 can similarly be positionally fixed to the bone, and in particular to the second portion 108 of the bone, with respect to relative movement along the longitudinal direction L, and in some examples with respect to any relative movement. In particular, the inner member 106 can include at least one inner member aperture, such as an inner member aperture 142, that is configured to receive an inner member bone anchor 141. The inner member aperture 142 can also be referred to as a third aperture 142 of the fixation device 100. Further, the inner member aperture 142 can be referred to as a distal aperture of the fixation device 100, and the at least one outer member aperture can be referred to as at least one proximal aperture of the fixation device 100 that is opposite the distal aperture along the proximal direction.

The inner member 106 can define at least one inner member aperture 142 that is configured to receive an inner member bone anchor 141 that fixes the inner member 106 to the second bone portion 108. The inner member bone anchor 141 can be a threaded bone fastener or bone screw, pin, rivet, nail, or adhesive. The inner member bone anchor 141 can be self-drilling in one example. The inner member bone anchor 141 can be locking screws. The inner member bone anchor 141 can be referred to as a third bone anchor of the fixation device 100. Similarly, the inner member aperture 142 can be referred to as a third aperture of the fixation device 100.

As described above, the inner member aperture 142 can be configured to receive the inner member bone anchor 141, and thus can be referred to as a bone fixation hole. The inner member aperture 142 can extend into or through the inner member 106 along a direction perpendicular to the central axis A₁ or the longitudinal direction L. In one example, the inner member aperture 142 defines a first end at a first location of the inner member 106 and a second end at a second location of the inner member 106 that is opposite the first location of the inner member 106, for instance with respect to the central axis A₁. Thus, the inner member aperture 142 can extend through the inner member 106 along a respective central axis. The central axis of the inner member aperture 142 can be parallel with the central axis of the outer member apertures 134 and 138. Alternatively, the central axis of the inner member aperture 142 can be angularly offset with respect to the central axis of the outer member apertures 134 and 138.

The inner member aperture 142 can be configured to receive the inner member bone anchor 141. In particular, the bone anchor 141 can be driven through a respective first location of the second bone portion 108 and into the inner member aperture 142 so as to fix the inner member 106 to the second bone portion 108 with respect to relative movement along the longitudinal direction L. In particular, the inner member bone anchor 141 can be driven at least into or through the inner member aperture 142. In one example, the inner member bone anchor 141 can be driven through the first location of the second bone portion 108, through the inner member aperture 142, and into a second location of the second bone portion 108. The second location of the second bone portion 108 can be opposite the first location of the second bone portion 108 along the central axis of the inner member aperture 142.

The inner member bone anchor 141 can threadedly engage the inner member 106 when the bone anchor 141 is driven into or through the aperture 142. In particular, the bone anchor 141 can threadedly engage an internal sidewall of the inner member 106 that defines the inner member aperture 142. The aperture 142 can be sized and shaped to receive the inner member bone anchor 141 such that the inner member 106 is substantially positionally fixed to the second bone portion 108 with respect to relative movement along the longitudinal direction L, and in some examples with respect to any relative movement. The inner member aperture can have a length in the longitudinal direction L that is about equal to an outer diameter of a threaded shaft of the inner member bone anchor 141 that threadedly engages the inner member 106. The inner member bone anchor 141 can be identically sized and shaped with respect to the outer member bone anchors 136 and 140. Similarly, the apertures 134, 138, and 142 can be identically sized and shaped. Thus, the bone anchors 136, 140, and 141 can be interchangeable in their respective apertures. Alternatively one or more of the bone anchors or apertures can be differently sized and/or shaped with respect to at least one other of the bone anchors or apertures as desired.

As described above, the outer member 102 can be movable with respect to the inner member bone anchor 141 along the central axis A₁ or the longitudinal direction L. Similarly, the inner member 106 can be movable with respect to the outer member bone anchors 136 and 140 along the central axis A₁ or the longitudinal direction L.

For instance, the outer member 102 can define an outer member slot 143 that is configured to receive the inner member bone anchor 141, such that the inner member bone anchor 141 is movable within the slot 143 along the longitudinal direction L. The slot 143 can have a length in the longitudinal direction L that is greater than its width in the lateral direction A. The outer member slot 143 can be referred to as a first slot of the fixation device 100. The outer member slot 143 can extend through the outer wall 114 of the outer member 102 at least one location, such as two locations of the outer wall 114 that are opposite each other with respect to the central axis A₁. The outer member slot 143 can have a length in the longitudinal direction L that is greater than an outer diameter the inner member bone anchor 141 when the third bone anchor 141 extends into the slot 143. The slot 143 can be elongate in the longitudinal direction L such that the outer member 102 is moveable in the longitudinal direction L relative to the inner member bone anchor 141. The inner member bone anchor 141 can be movable relative to the slot 143 from a first position when the fixation device 100 is in a loaded configuration (described below with respect to FIG. 6) to a second position when the fixation device 100 is in a compressed configuration (described below with reference to FIG. 2). The second position of the bone anchor 141 can be spaced from the first position of the bone anchor 141 in the distal direction. The outer member 102 can be movable relative to the bone anchor 141 while the third bone anchor 141 is fixed to the second bone portion 108.

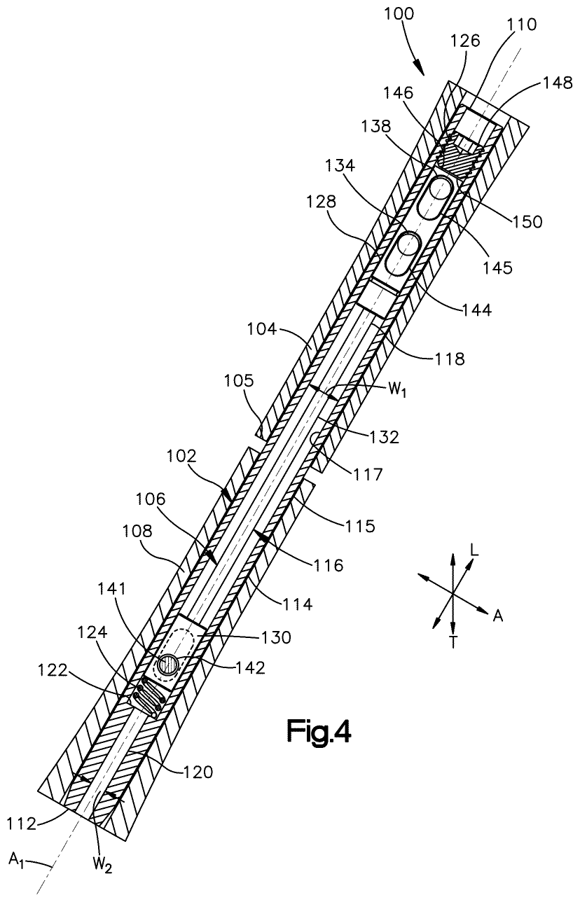

In some examples, a side wall of the outer member 102 that defines the outer member slot 143 can include proximal and distal ends that are configured to contact the bone anchor 141, thereby providing respective stop surfaces with respect to movement in the proximal direction and movement in the distal direction, respectively. Thus, the bone anchor 141 can travel within the outer member slot 143 in the proximal direction until the bone anchor 141 contacts a proximal stop surface of the side wall of the outer member 102, and can travel within the outer member slot 143 in the distal direction until the bone anchor 141 contacts the distal stop surface of the side wall of the outer member 102. In some examples, when the fixation device 100 is in the initial configuration (FIG. 3), the bone anchor 141 can be disposed in a corresponding initial position adjacent or against the proximal stop surface of the outer member 102. When the fixation device 100 is in the loaded configuration (FIGS. 4-6), the bone anchor 141 can be disposed in a respective first position adjacent or against the distal stop surface of the outer member 102. When the fixation device 100 is in the compressed configuration shown at FIG. 2, the inner member bone anchor 141 can be disposed in a respective second position at any location from the distal end to the proximal end.

The inner member bone anchor 141 can be driven through the outer member slot 143 when it is driven through the third aperture 142. The inner member bone anchor 141 can thus be configured to extend through each of the first slot 143 and the third aperture 142. A portion of the inner member aperture 142 can be aligned with at least a portion of the outer member slot 143 along the longitudinal direction L. The inner member aperture 142 can also be aligned with the outer member slot 143 along the transverse direction T. The central axis of the third aperture 142 can be parallel to a central axis of the outer member slot 143. In some examples, the central axis of the inner member aperture 142 can be coaxial with a central axis of the outer member slot 143. The outer member slot 143 can have a length along the longitudinal direction L that is greater than the length of the inner member aperture 142 along the longitudinal direction L. The outer member 102 can be movable in the longitudinal direction L relative to the inner member bone anchor 141 while the inner member 106 is positionally fixed to the third bone anchor 141 with respect to relative movement along the longitudinal direction L.

The outer member 102 can include a channel 116 adapted to receive the inner member 106. The inner member 106 can be movably received in the channel 116. The inner member 106 can be movable relative to the outer member 102 in the channel 116. The channel 116 can be defined by the inner surface 117 of the outer wall 114. The channel 116 can be elongate along the central axis A₁. The channel 116 can extend from the first end 110 toward the second end 112. The channel 116 can extend from the first end 110 to the second end 112. The channel 116 can extend through the first end 110 and the second end 112.

The channel 116 can include a first channel portion 118 and a second channel portion 120. The inner member 106 can be positioned in the first channel portion 118. The first channel portion 118 and the second channel portion 120 can have different widths. The first channel portion 118 can have a first width W₁. The first width W₁ can be measured in a plane including the transverse direction T and the lateral direction A. The second channel portion 120 can have a second width W₂. The first width W₁ can be greater than the second width W₂. A ratio of the first width W₁ to the second width W₂ can be about 1:1, about 2:1, about 3:1, about 4:1, or about 5:1.

The first channel portion 118 can have a first length L₁ (FIG. 3). The first length L₁ can be greater than the first width W₁. The second channel portion 118 can have a second length L₂. The second length L₂ can be greater than the second width W₂. In some examples, the first length L₁ can be greater than the second length L₂. In other examples, the first length L₁ can be equal to the second length L₂. A ratio of the first length L₁ to the second length L₂ can be about 1:1, about 2:1, about 3:1, about 4:1, or about 5:1. The outer wall 114 can have a thickness between the outer surface 115 and the inner surface 117. The thickness of the outer wall 114 at the second channel portion 120 can be greater than the thickness of the outer wall 114 at the first channel portion 118. A spring seat 122 can separate the first channel portion 118 from the second channel portion 120. In some examples, the spring seat 122 can be a shoulder.

The inner member 106 can be adapted to be positioned in the channel 116. The inner member 106 can be positioned in the first portion 118 of the channel 116. The inner member 106 can include a first portion 128 and a second portion 130. The first portion 128 can be proximal to the second portion 130. The first portion 128 can be opposite the second portion 130 along the longitudinal direction L. The first portion 128 can be spaced from the second portion 130 along the longitudinal direction L. The first portion 128 can be spaced from the second portion 130 along the central axis A₁. A width of the first portion 128 can be equal to a width of the second portion 130 in the lateral direction A. In other examples, the width of the first portion 128 is greater than the width of the second portion 130. In other examples, the width of the first portion 128 is less than the width of the second portion 130. The width of the first and second portions 128 and 130 are each smaller than the first width W₁ of the first portion 118 of the channel 116. The second portion 130 can have a length in the longitudinal direction L that is less than a length of the first portion 128. The first and second portions 128 and 130 can each have a length in the longitudinal direction L. In some examples, the length of the second portion 130 is greater than the length of the first portion 128. In other examples, the length of the second portion 130 is equal to the length of the first portion 128. In other examples, the length of the second portion 130 is less than the length of the first portion 128.

In some examples, the first portion 128 extends directly from the second portion 130. In other examples, a central portion 132 can extend from the first portion 128 to the second portion 130. A width of the central portion 132 can be smaller than a width of the first portion 128. A width of the central portion 132 can be smaller than a width of the second portion 130. The widths can be measured along the lateral direction A. The central portion 132 can have a length in the longitudinal direction L. The length of the central portion 132 can be greater than a length of the first portion 128. The length of the central portion 132 can be greater than a length of the second portion 130. The length of the central portion 132 can be at least about 30%, about 40%, about 50%, about 60%, about 70%, about 80% or about 90% of the length of the outer member 102.

The inner member 106 can include at least one inner member slot that is configured to receive the respective at least one outer member bone anchor. In one example, the at least one inner member slot can include a first inner member slot 144 configured to receive the first outer member bone anchor 136, and a second inner member slot 145 configured to receive the second outer member bone anchor 140. The first and second inner member slots 144 and 145 can be referred to as second and third slots, respectively, of the fixation device 100. The first and second inner member slots 144 and 145 can extend through the inner member 106 along any suitable direction, such as a direction that is perpendicular to the longitudinal direction L. In one example, the first and second inner member slots 144 and 145 can extend from a first side of the inner member 106 toward a second side of the inner member 106 without extending completely through the inner member 106. In other examples, the first and second inner member slots 144 and 145 can extend completely through the inner member 106. The first and second inner member slots 144 and 145 can be elongate in the longitudinal direction L.

The first inner member slot 144 can be elongate along the longitudinal direction L such that the inner member 106 is movable along the longitudinal direction L relative to the first outer member bone anchor 136 when the bone anchor 136 extends at least into or through the first inner member slot 144. In some examples, a first side wall of the inner member 106 that defines the first inner member slot 144 can include first proximal and distal ends that are configured to contact the first outer member bone anchor 136, thereby providing respective first stop surfaces with respect to movement of the bone anchor 136 in the slot 144 in the proximal direction and movement of the bone anchor 136 in the slot 144 in the distal direction, respectively. Thus, the bone anchor 136 can travel within the first inner member slot 144 in the proximal direction until the bone anchor 136 contacts the first proximal stop surface of the first side wall of the inner member 106, and can travel within the first inner member slot 144 in the distal direction until the bone anchor 136 contacts the second stop surface of the side wall of the inner member 106. In this regard, the bone anchor 136 defines a stop surface with respect to travel in the first inner member slot 144 in both the proximal and distal directions. In some examples, when the fixation device 100 is in the loaded configuration (FIG. 6), the bone anchor 136 can be disposed in a corresponding first position adjacent or against the proximal stop surface of the inner member 106. When the fixation device 100 is in the compressed configuration shown at FIG. 2, the bone anchor 136 can be disposed in a respective second position at any location in the slot 144 from the distal end to the proximal end. For instance, in some examples the bone anchor 136 can contact the distal stop surface of the sidewall defining the slot 144 in the second position. In other examples, the bone anchor 136 can be spaced from the distal end of the sidewall defining the second slot 144 in the second position. The bone anchor 136 can move in the slot 144 along the longitudinal direction L as the outer member 102 moves relative to the inner member 106 along the longitudinal direction L. Thus, the bone anchor 136 can move in the slot 144 along the longitudinal direction L as the first bone portion 104 moves relative to the second bone portion 106 along the longitudinal direction L.

With continuing reference to FIG. 2, the second inner member slot 145 can be configured to receive the second outer member bone anchor 140. The second inner member slot 145 can be elongate along the longitudinal direction L such that the inner member 106 is movable along the longitudinal direction L relative to the second outer member bone anchor 140 when the bone anchor 140 extends at least into or through the second inner member slot 145. In some examples, a second side wall of the inner member 106 that defines the second inner member slot 145 can include second proximal and distal ends that are configured to contact the second outer member bone anchor 140, thereby providing respective second stop surfaces with respect to movement of the bone anchor 140 in the slot 145 in the proximal direction and movement of the bone anchor 140 in the slot 145 in the distal direction, respectively. Thus, the bone anchor 140 can travel within the second inner member slot 145 in the proximal direction until the bone anchor 140 contacts the second proximal stop surface, and can travel within the second inner member slot 145 in the distal direction until the bone anchor 140 contacts the second distal stop surface. In some examples, when the fixation device is in the loaded position (FIG. 6), the bone anchor 140 can be disposed in a corresponding first position adjacent or against the second proximal stop surface of the outer member 102. When the fixation device 100 is in the compressed configuration shown at FIG. 2, the second outer member bone anchor 140 can be disposed in a respective second position at any location in the slot 145 from the distal end to the proximal end. For instance, in some examples the bone anchor 136 can contact the distal stop surface of the sidewall defining the slot 144 in the second position. In other examples, the bone anchor 140 is spaced from the distal end of the sidewall defining the slot 145 in the second position. The bone anchor 140 can move in the slot 145 along the longitudinal direction L as the outer member 102 moves relative to the inner member 106 along the longitudinal direction L. Thus, the bone anchor 140 can move in the slot 145 along the longitudinal direction L as the first bone portion 104 moves relative to the second bone portion 106 along the longitudinal direction L. While the inner member 106 is illustrated as defining the first and second inner member slots 144 and 145 as described above, it should be appreciated that the first and second inner member slots 144 and 145 can be replaced by a single slot that receives each of the bone anchors 136 and 140. The inner member 106 can move relative to the outer member 102 in the distal direction from an initial position when the fixation device 100 is in the initial configuration (FIG. 3) to a first position when the fixation device 100 is in the loaded configuration (FIGS. 4-6). The inner member 106 can move relative to the outer member 102 in the proximal direction from the first position when the fixation device 100 is in the loaded configuration (FIGS. 4-6) to a second position when the fixation device 100 is in the compressed configuration. The second position of the inner member 106 can be offset from the initial position in the distal direction. As described above, at least one of the outer and inner members 102 and 106 can be movable relative to the other of the outer and inner members 102 and 106 so as to decrease a distance from the first outer member aperture 134 to the inner member aperture 142 along the central axis A₁, and thus along the longitudinal direction L. Even if the apertures 134 and 142 are circumferentially offset with respect to each other, the distance is measured along the central axis of the fixation device 100 and thus along the longitudinal direction L.

The inner member 106 can define a first end 146. The first end 146 can define a proximal end of the inner member 106. The first end 146 of the inner member 106 can be spaced from the first end 110 of the outer member 102 by an initial distance D₁ along the longitudinal direction L when the fixation device 100 is in the initial configuration. The first end 146 of the inner member 106 can be spaced from the first end 110 of the outer member 102 by a first distance D₁ when the fixation device 100 is in the loaded configuration. The first end 146 of the inner member 106 can be spaced from the first end 110 of the outer member 102 by a second distance D₂ when the fixation device 100 is in the compressed configuration. The initial distance D₁ can be less than the first distance D₁. The first distance D₁ can be greater than the second distance D₂. The inner member 106 can in the distal direction relative to the outer member 102 from the initial configuration to the loaded configuration. The inner member 106 can in the proximal direction relative to the outer member 102 from the loaded configuration to the compressed configuration.

The fixation device 100 can include a biasing member 124 adapted to apply a compression force against the outer member 102 and the inner member 106 that biases either or both of the outer member 102 and the inner member 106 relative to the other of the outer member 102 and the inner member along the longitudinal direction. In particular, the biasing member 124 can apply the compression force to the inner member 106 that biases the inner member 106 in the proximal direction relative to the outer member 102. The compression force can also be allied to the to the outer member 102 that biases the outer member 102 in the distal direction relative to the inner member 106. The biasing member 124 can be any suitable resilient element. For instance, the biasing member 124 can be a spring. Alternatively, the biasing member 124 can be an intrinsically resilient material, such as rubber. In some examples, the biasing member 124 can be a shape memory material. The biasing member 124 can be made from nickel-titanium (Nitinol) such that the biasing member 124 expands as it is heated. The biasing member 124 can be compressed gas. The biasing member 124 can be deformable, such as compressible. The biasing member 124 can exert a force in a range of approximately 30 to 150 pounds per inch of compression, such as a range of approximately 50 to 100 pounds per inch of compression, such as approximately 80 pounds per inch of compression.

The biasing member 124 can be seated against the outer member 102, and can extend from the outer member 102 to the inner member 106. In one example, the biasing member 124 can be disposed in the channel 116 of the outer member 102. For instance, the biasing member 124 can be positioned in the first channel portion 118. The biasing member 124 can be positioned between the spring seat 122 and the inner member 106. The biasing member 124 can be compressed or otherwise deformed between the outer member 102 and the inner member 106 when the fixation device 100 is in the loaded configuration (FIGS. 4-6) and when the fixation device is in the compressed configuration (FIG. 2). The biasing member 124 can urge the inner member 106 in the proximal direction toward the first end 110 of the outer member 102. The biasing member 124 can cause relative movement between the outer member 102 and the inner member 106 along the longitudinal direction L. The biasing member 124 can move the inner member 106 relative to the outer member 102 such that 1) the first outer member bone anchor 136 moves relative to the first inner member slot 144, 2) the second outer member bone anchor 140 moves relative to the second inner member slot 145, and 3) the inner member bone anchor 141 moves relative to the outer member slot 143. In other examples, the biasing member 124 can be configured so as to be placed in tension between the outer member 102 and the inner member 106. Further, instead of urging the inner member 106 to move relative to the outer member 102 in the proximal direction, the biasing member 124 can urge the outer member 102 to move relative to the inner member 106 in the distal direction. Alternatively still, the biasing member 124 can urge both 1) the inner member 106 to move relative to the outer member 102 in the proximal direction, and 2) the outer member 102 to move relative to the inner member 106 in the distal direction.

The first bone portion 104 and the second bone portion 108 can be adjacent to each other along the longitudinal direction L. For instance, the first bone portion 104 can be adjacent the second bone portion 108 in the proximal direction. The second bone portion 108 can be adjacent the first bone portion 104 in the distal direction. When the fixation device 100 is in the compressed configuration shown in FIG. 2, either of the first and second bone portions 104 108 can be biased toward or against each other in response to the compression force applied by the biasing member 124. Thus, the first bone portion 104 and second bone portion 108 exert a compressive force against each other to promote healing. In particular, the first bone portion 104 can be urged toward the second bone portion 108 as the first outer member bone anchor 136 moves in the first inner member slot 144 from the respective first position to the respective second position. Similarly, the first bone portion 104 can be urged toward the second bone portion 108 as the second outer member bone anchor 140 moves in the second inner member slot 145 from the respective first position to the respective second position. The second bone portion 108 can be urged toward the first bone portion 104 as the inner member bone anchor 141 moves in the outer member slot 143 from the respective first position to the respective second position. The bone anchors 136, 140, and 141 can simultaneously move within the respective slots 144, 145, and 143.

The fixation device 100 can include an actuator 126 that is configured to actuate the biasing member 124 to a loaded position whereby the fixation device 100 assumes the loaded configuration. In particular, the actuator 126 can deliver a loading force to the inner member 106 that causes the biasing member 124 to deflect from its initial position to its loaded position. In one example, the loading force applied by the actuator 126 can cause the inner member 106 to move relative to the outer member 102. The inner member 106, in turn, can cause the biasing member 124 to move from its initial position to its loaded position. In particular, the inner member 106 can cause the biasing member 124 to be compressed along the longitudinal direction L, and in particular along the distal direction. Thus, it can be said that the actuator 126 can move in a respective first direction with respect to the outer member 102 from its initial position to its loaded position. The first direction of the actuator 126 can be defined by the distal direction. Once the biasing member 124 has been moved to its loaded position as shown in FIGS. 4-6, movement of the actuator 126 in a respective second direction relative to the outer member 102, opposite the respective first direction, can iterate the fixation device from the loaded configuration shown in FIGS. 4-6 to the compressed configuration shown in FIG. 2, which causes the biasing member 124 to iterate to a compressed position, whereby the biasing member 124 applies the biasing force to the inner member 106 in the second direction relative to the outer member 102, and the fixation member 100 assumes the compressed configuration. The second direction can be defined by the proximal direction.

The actuator 126 can transition the biasing member 124 from a first state (FIG. 3) to a second state (FIGS. 4-6) as the actuator moves in the first direction. The first state can be defined by the initial position, and the second state can be defined by the loaded position. In some examples, the biasing member 124 does not exert a force against the inner member 106 in the first state. In other examples, the biasing member 124 exerts a force against the inner member 106 in the first state but the force is insufficient to move the inner member 106 relative to the outer member 102. Movement of the actuator 126 in the first direction can transition the biasing member 124 from the first state to the second state. In some examples, the actuator 126 transitions the biasing member 124 from the first state to the second state after the fixation element 100 is implanted in the intramedullary canal. In other examples, the actuator 126 transitions the biasing member 124 from the first state to the second state before the fixation device 100 is implanted. The biasing member 124 can exert a force sufficient to move the inner member 106 relative to the outer member 102 when the biasing member 124 is in the second state. The actuator 126 can apply a counterforce to the biasing member 124 when the biasing member 124 is in the second state, which prevents the biasing member 124 from exerting a force against the outer member 102 and inner member 106 sufficient to move either of the bone portions 104 and 108. When the actuator is moved in the second direction, which can be away from the biasing member 124, the counterforce is removed. Thus, the biasing member 124 moves from the second state to a third state whereby the biasing member 124 exerts a force against each of the outer member 102 and the inner member 106 sufficient to move the inner member 106 and the outer member 102 relative to each other. Thus, when the outer member 102 is fixed to the first bone portion 104 and the inner member 106 is fixed to the second bone portion 108, the biasing member applies a continuous compression force to either or both of the first and second bone portions 104 and 108 against each other.

The actuator 126 and biasing member 124 can be disposed on opposite sides of the inner member 106. In one example, the biasing member 124 is distal of the inner member 106, and the actuator 126 is proximal of the inner member 106. The actuator 126 and biasing member 124 can be disposed on opposite sides of the inner member 106 along the central axis A₁. The actuator 126 can move toward the biasing member 124 as the actuator 126 moves in the first direction. The actuator 126 can move away from the biasing member 124 as the inner member 106 moves in the second direction.

In one example, the actuator 126 can be disposed in the channel 116 of the outer member 102, and threadedly coupled to the outer member 102, such that the actuator is aligned with the inner member 106 along the lateral direction. In one example, the actuator can be threadedly coupled to the inner surface 117 of the outer wall 114 of the outer member 102. Thus, the actuator 126 can be adapted to rotate relative to the outer member 102, which causes the actuator 126 to move selectively in the first or distal direction, and in the second or proximal direction relative to the outer member 102. For instance, rotation of the actuator 126 in a first direction of rotation can cause the actuator 126 to move in the first direction relative to the outer member 102, which can iterate the fixation device 100 from the initial configuration to the loaded configuration. In particular, the actuator 126 can bear against the inner member 106, such that movement of the actuator 126 in the first direction drives the inner member 106 to move in the first direction, which causes the biasing member 124 to compress to its loaded position. Rotation of the actuator 126 in a second direction of rotation opposite the first direction of rotation can cause the actuator 126 to move in the second direction relative to the outer member 102, which iterates the fixation device 100 from the loaded configuration to the compressed configuration. In particular, movement of the actuator 126 in the second direction can allow the force of the biasing member 124 to drive the inner member to move in the second direction, and also allow the biasing member 124 to drive the outer member 102 to move in the first direction.

The actuator 126 can include a proximal end 148 and a distal end 150 opposite the proximal end 148. As described above, the actuator 126 can contact the inner member 106 as the actuator 126 moves toward the first position. In particular, the distal end 150 can contact the inner member 106 as the actuator 126 moves toward the first position. For instance, the distal end 150 of the actuator 126 can bear against the first end 146 of the inner member 106 as the actuator 126 moves toward the first position. The proximal end 148 can be distal of the first end 110 of the outer member 102 when the actuator is in an actuated position that drives the biasing member 124 to its loaded position. The distal end 150 of the actuator 126 can disengage from the inner member 106 as the actuator 126 moves in the second direction from the actuated position to a second position. In some examples, the inner member 106 can contact the actuator 126 as the biasing member 124 moves the inner member 106 in the second direction relative to the outer member 102. In some examples, the inner member 106 remains spaced from the actuator 126 when the actuator 126 is in the second position, as shown in FIG. 2. When the actuator 126 has been moved from the first position to the second position, the fixation device can be placed in the compressed configuration. When the actuator 126 is spaced from the inner member 106 along the longitudinal direction L, the biasing member 124 can allow continuous compression of the first and second bone portions 104 and 108 against each other.

The actuator 126 can include an engagement feature 154 (FIG. 3). The engagement feature 154 can be adapted to be engaged by a tool to move the actuator. The engagement feature 154 can be a recess adapted to receive a tool (e.g., screwdriver or Allen wrench). The engagement feature 154 can be a protrusion adapted to engage a tool (e.g., socket wrench). The actuator 126 can be moved by the tool engaged with the engagement feature 154.

While the actuator 126 has been described as being threadedly or otherwise translatably secured to the outer member 102 with respect to movement of the actuator 126 relative to the outer member 102 along the longitudinal direction L, it should be appreciated that the actuator 126 can be alternatively constructed as desired. For instance, the actuator can be defined by an external device, such as a plunger, that can apply an external force to the inner member 106 that drives the inner member 106 against the biasing member 124 to iterate the fixation device 100 from the initial configuration to the loaded configuration in the manner described above. Removal of the external force, for instance by removing the plunger from the inner member 106, can cause the fixation device 100 to iterate from the loaded configuration to the compressed configuration as described above.

A method of providing compression across the first and second bone portions 104 and 108 with the fixation device will now be described with initial reference to FIG. 3. The method can include the step of inserting the fixation device 100 into the intramedullary canal of the bone such that the outer member 102 spans across a bone gap 105 from the first bone portion 104 to the second bone portion 108. The bone gap 105 can be a fracture, for instance due to trauma or osteoetomy, or can be an anatomical bone gap that is to be corrected. The fixation device 100 can be in the initial configuration when inserted into the medullary canal. Thus, the biasing member 124 can be in its initial position when the fixation device 100 is inserted into the medullary canal.

As shown at FIG. 3, after the fixation device 100 has been inserted into the medullary canal, the inner member 106 can be positionally fixed to the second bone portion 108. For instance, the inner member bone anchor 141 can be driven through the second bone portion 108 and the inner member aperture 142, thereby positionally fixing the inner member 106 to the second bone portion 108. The inner member bone anchor 141 is further driven into or through the outer member slot 143. In one example, the inner member bone anchor 141 can be driven along the transverse direction T. In particular, the inner member bone anchor 141 can be spaced from the distal end of the outer member slot 143 so as to allow for future movement of the inner member bone anchor 141, and thus the inner member 106, in the distal direction relative to the outer member 102. For instance, the inner member bone anchor 141 can abut or can be adjacent the proximal end of the outer member slot 143. The inner member bone anchor 141 can be driven through a pre-drilled hole in the second bone portion 108, or the bone anchor 141 can be self-tapping. When the fixation device 100 is inserted into the medullary canal, the bone gap 105 between the first and second bone portions 104 and 108 can be reduced such that the bone portions 104 and 108 are adjacent each other or abut each other, thereby reducing or eliminating the bone gap 105.

Next, referring to FIG. 4, the biasing member 124 can be actuated from the initial position to the loaded position. In particular, the actuator 126 can be driven in the first or distal direction, which in turn drives the inner member 106 to translate in the distal direction. Translation of the inner member 106 in the distal direction can compress the biasing member 124, thereby iterating the biasing member 124 from the initial position to the loaded position. Because the inner member 106 is positionally fixed to the second bone portion 108 of the inner member 106 in the distal direction also causes the second bone portion 108 to translate in the distal direction relative to the first bone portion 106, thereby increasing the bone gap 105 between the first bone portion 104 and the second bone portion 108 along the longitudinal direction L. Furthermore, movement of the inner member 106 in the distal direction causes the inner member bone anchor 141 to translate in the distal direction within the outer member slot 143. The actuator 126 can be driven in the first or distal direction until the inner member bone anchor 141 abuts the distal stop surface of the outer member slot 143. Alternatively, the actuator 126 can be driven in the first or distal direction until the inner member bone anchor 141 is at a location spaced from the distal stop surface of the outer member slot 143 in the proximal direction.

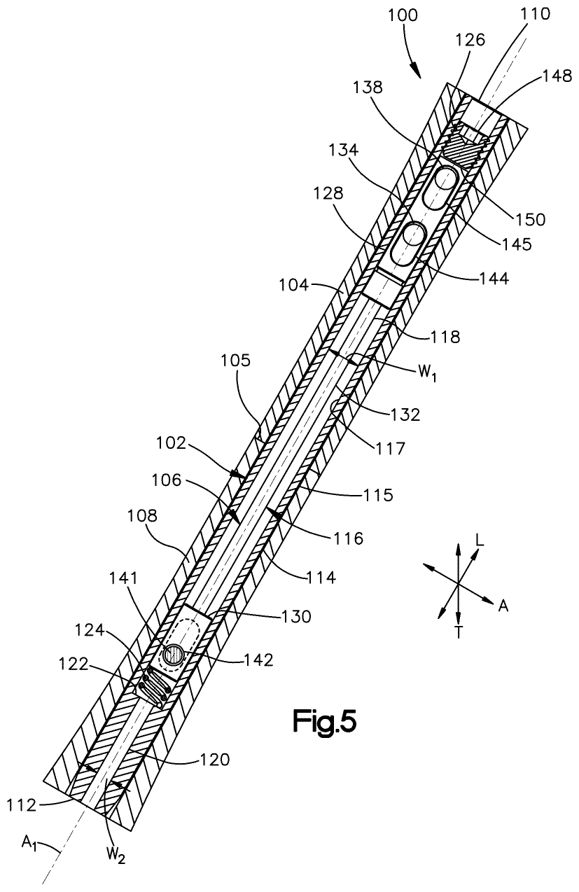

Next, referring to FIG. 5, the bone gap 105 between the first and second bone portions 104 and 108 can be reduced. In particular, the first bone portion 104 can be driven in the distal direction toward the second bone portion 108 until the first bone portion 104 is disposed adjacent the second bone portion 108 or abuts the second bone portion, thereby reducing or eliminating the bone gap 105. Therefore, the inner member 106 is fixed to the second bone portion 108, the bone gap 105 has been reduced or eliminated, and the biasing member 124 is in the loaded position. In other examples, the fixation device 100 can be pre-loaded whereby the actuator 126 is driven in the distal direction as described above to iterate the biasing member 124 from its initial position to its loaded position, and then the fixation device can be subsequently inserted into the medullary canal as described above. Next, the inner member bone anchor 141 can be driven through the second bone portion 108, the inner member aperture 142, and into or through the outer member slot 143, in the manner described above.

Referring now to FIG. 6, once the fixation device 100 is disposed in the intramedullary canal in the loaded configuration with the outer member 102 positionally fixed to the second bone portion 108 and the bone gap 105 reduced, the outer member 102 can be positionally fixed to the first bone portion 104. For instance, the at least one outer member bone anchor can be driven through the first bone portion 104 and the respective at least one outer member aperture 142, thereby fixing the outer member 102 to the first bone portion 104. The at least one outer member bone anchor can also be driven into or through the respective at least one inner member slot. Each at least one outer member bone anchor can be driven through a pre-drilled hole through the first bone portion 104, or can be self-tapping as desired.

In particular, the first outer member bone anchor 136 can be driven through the first bone portion 104 and the first outer member aperture 134, thereby positionally fixing the outer member 102 to the first bone portion 104. The first outer member bone anchor 136 is further driven into or through the first inner member slot 144. In one example, the first outer member bone anchor 136 can be driven along the transverse direction T. In particular, the first outer member bone anchor 136 can be spaced from the distal end of the first inner member slot 144 in the proximal direction so as to allow for future movement of the first outer bone anchor 136, and thus the inner member 106, in the proximal direction relative to the outer member 102. For instance, the first outer bone anchor 136 can abut or can be adjacent the proximal end of the first inner member slot 144.

Next, the second outer member bone anchor 140 can be driven through the first bone portion 104 and the second outer member aperture 138, thereby positionally fixing the outer member 102 to the first bone portion 104. The second outer member bone anchor 140 is further driven into or through the second inner member slot 145. In one example, the second outer member bone anchor 140 can be driven along the transverse direction T. In particular, the second outer member bone anchor 140 can be spaced from the distal end of the second inner member slot 145 in the proximal direction so as to allow for future movement of the second outer bone anchor 140, and thus the inner member 106, in the proximal direction relative to the outer member 102. For instance, the second outer bone anchor 140 can abut or can be adjacent the proximal end of the second inner member slot 144. While the first outer member bone anchor 136 has been described as implanted prior to the second outer member bone anchor 140, it should be appreciated that the second outer member bone anchor 140 can alternatively be implanted prior to the first outer member bone anchor 140. Alternatively, only one of the bone anchors 136 and 140 can be implanted.

Once the inner member 106 has been positionally fixed to the second bone portion 108, the outer member 102 has been positionally fixed to the first bone portion 104, and the fixation device 100 is in the loaded configuration, the fixation device can then be iterated to the compressed configuration. In particular, referring now to FIG. 2, the actuator 126 can be driven in the second or proximal direction in the manner described above. Thus, the actuator 126 is moved in a direction away from the biasing member 124, which causes the biasing member to apply the compression force against the inner member 106 in the proximal direction to drive the inner member 106 in the proximal direction. This, in turn, biases the second bone portion 108 toward the first bone portion 104 in the proximal direction. Further, because the biasing member 124 is also seated against the outer member 102, the compression force can be applied to the outer member 102 in the distal direction to drive the outer member 102 in the distal direction. This, in turn, biases the first bone portion 104 toward the second bone portion in the distal direction. Biasing the first and second bone portions 104 and 108 toward each other can close the bone gap 105 if it is open, and can apply constant compression to the first and second bone portions 104 and 108 across the bone gap 105, thereby promoting healing. As the inner member 106 is driven in the proximal direction and the outer member 102 is driven in the distal direction, 1) the first and second outer member bone anchors 136 and 140 can be driven to move in the proximal direction in the respective first and second inner member slots 144 and 145, and 2) the inner member bone anchor 141 can be driven to move in the distal direction in the outer member slot 143.

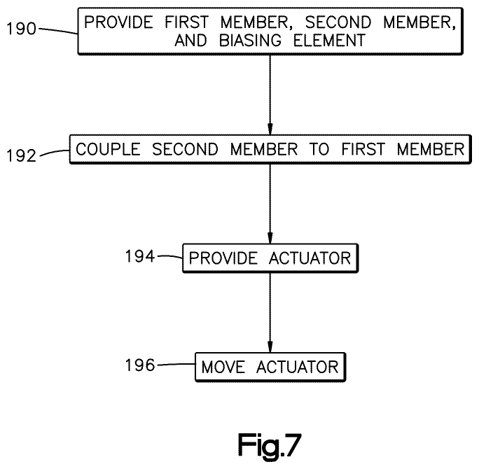

Referring now to FIG. 7, a method of assembling the fixation device 100 can include a step 190 of providing the outer member 102 and the inner member 106. In some examples, step 190 can include providing the biasing member 124 and positioning the biasing member 124 in the channel 116. The method can include a step 192 of coupling the inner member 106 to the outer member 102. The coupling step 192 can include positioning the inner member 106 in the channel 116 of the outer member 102. The coupling step 192 can include positioning the biasing member 124 in the channel 116. The coupling step can include moving the inner member 106 into contact with the biasing member 124.

The method can include a step 194 of providing the actuator 126. The step 194 can include engaging the outer member 102 with the actuator 126.

The method can include a step 196 of moving the actuator 126. The moving step 196 can include moving the actuator 126 in the first direction. The moving step 104 can include moving the inner member 106 relative to the outer member 102. The moving step 196 can include transitioning the biasing member 124 from the first state to the second state. The moving step 196 can include aligning the outer member slot 143 with the inner member aperture 142 along the transverse direction T. The moving step 196 can include aligning the each of the first and second outer member apertures 134 and 138 with the respective first and second inner member slots 144 and 145 along the transverse direction T. The moving step 196 can include engaging the actuator 126 with an insertion and/or actuation tool. The moving step 196 can include rotating the actuator 126.

While systems and methods have been described in connection with the various embodiments of the various figures, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

When values are expressed as approximations by use of the antecedent “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function, and the person skilled in the art will be able to interpret it as such. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, reference to values stated in ranges includes each and every value within that range.

Throughout this document, words are to be afforded their normal meaning as would be understood by those skilled in the relevant art. However, so as to avoid misunderstanding, the meanings of certain terms are specifically defined or clarified.

It is to be appreciated that certain features are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present disclosure. Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

What is claimed:

1. An intramedullary nail that extends along a central axis, the intermedullary nail comprising:
a first member configured to be positioned in a medullary canal of a bone, wherein the first member defines at least one first bone fixation hole configured to receive a respective at least one first bone fixation element so as to couple the first member to a first bone portion of the bone;
a second member configured to be at least partially inserted into the first member, wherein the second member defines at least one second bone fixation hole configured to receive a respective at least one second bone fixation element so as to couple the second member to a second bone portion of the bone that is spaced from the first bone portion;
a biasing member adapted to apply a biasing force to at least one of the first and second members so as to decrease a distance from the at least one first bone fixation hole to the at least one second bone fixation hole along the central axis.

2. The intramedullary nail of claim 1, wherein the first member is configured to be fixed to the first bone portion by a first bone fixation element, and the second member is configured to be fixed to the second bone portion by a second bone fixation element.

3. The intramedullary nail of claim 1, wherein the first member includes a first slot that is elongate along a longitudinal axis of the first member and the second bone fixation element is positioned within the first slot such that the first member is movable relative to the second bone fixation element.

4. The intramedullary nail of claim 3, wherein the second member includes a second slot that is elongate along a longitudinal axis of the second member and the first bone fixation element is positioned within the second slot such that the second member is movable relative to the first bone fixation element.

5. The intramedullary nail of claim 1, wherein the first member includes a channel, and a first end of the biasing member is supported by the first member in the channel, and a second end of the biasing member bears against the second member in a select direction, and the biasing force is applied to the second member in the select direction.

6. The intramedullary nail of claim 5, wherein the biasing member extends from a shoulder of the first member inside the channel to the second member in the select direction.

7. The intramedullary nail of claim 1, wherein the first member includes a channel adapted to receive the second member and the biasing member.

8. The intramedullary nail of claim 1, wherein the intramedullary nail is configured to transition from an initial configuration to a compressed configuration,
wherein in the initial configuration a distal end of the first member is spaced from a distal end of the second member by an initial distance, and
wherein in the compressed configuration the distal end of the first member is spaced from the distal end of the second member by a compressed distance.

9. The intramedullary nail of claim 8, wherein the initial distance is greater than the compressed distance.

10. The intramedullary nail of claim 8, wherein a length of the intramedullary nail is the same in the initial configuration and the compressed configuration, wherein the length is measured along the central axis from a first terminal end of the first member to a second terminal end of the second member opposite the first terminal end.

11. The intramedullary nail of claim 5, wherein the biasing member is compressed between the first member and the second member.

12. The intramedullary nail of claim 5, further comprising an actuator adapted to move the second member relative to the first member against the biasing force of the biasing member.

13. The intramedullary nail of claim 12, wherein the actuator is adapted to move the second member relative to the first member along the longitudinal axis.

14. The intramedullary nail of claim 12, wherein the actuator is threadedly engaged with the first member, and rotational motion of the actuator moves the second member linearly relative to the first member.

15. The intramedullary nail of claim 12, wherein the actuator is within the channel of the first member.

16. The intramedullary nail of claim 15, wherein the channel is elongate along the longitudinal axis.

17. The intramedullary nail of claim 16, wherein movement of the actuator in a first direction transitions the biasing member from an initial configuration to an energized configuration.

18. The intramedullary nail of claim 17, wherein movement of the actuator in a second direction allows the second member to move relative to the first member.

19. The intramedullary nail of claim 18, wherein the biasing force is applied to the second member, and the actuator is movable away from the second member so as to allow the biasing force to move the second member so as to decrease the distance between the at least one first bone fixation hole to the at least one second bone fixation hole.

20. The intramedullary nail of claim 18, wherein the second direction is opposite the first direction.
